(19) United States
(12) Patent Application Publication
(10) Pub. No.: US 2007/0027387 A1
(43) Pub. Date: Feb. 1, 2007

(54) Integrated carrier for providing support, templates and instructions for biopotential electrode array

(75) Inventor: Charles Fendrock, Sudbury, MA (US)

Correspondence Address: Mark J. Pandiscio;Pandiscio & Pandiscio, P.C., 470 Totten Pond Road, Waltham, MA, 02451-1914, US

(73) Assignee: NeuroMetrix, Inc.

(21) Appl. No.: 11/494,174

(22) Filed: Jul. 27, 2006

Related U.S. Application Data
(60) Provisional application No. 60/703,076, filed on Jul. 28, 2005.

(51) Int. Cl. A61B 5/04

(52) U.S. Cl. 600/372; 600/382

(57) ABSTRACT

An integrated carrier for a biopotential electrode array, the integrated carrier comprising: a substantially planar, sheet-like body which is formed out of a material which is sufficiently stiff as to provide mechanical support to the electrode array during shipment and storage; at least one securing unit disposed on the body for securing the biopotential electrode array to the body; and at least one template disposed in the body for guiding proper placement of the electrode array against the patient. In another form of the invention, there is provided an integrated carrier for a biopotential electrode array, the integrated carrier comprising: a substantially planar, sheet-like body which is formed out of a material which is sufficiently stiff as to provide mechanical support to the electrode array during shipment and storage; at least one securing unit disposed on the body for securing the biopotential electrode array to the body; at least one template disposed in the body for guiding proper placement of the electrode array against the patient; and instructions for use, wherein the instructions are printed on the body.

### REFERENCE TO PENDING PRIOR PATENT APPLICATION

This patent application claims benefit of pending prior U.S. Provisional Patent Application Ser. No. 60/703,076, filed Jul. 28, 2005 by Charles Fendrock for INTEGRATED CARRIER AND TEMPLATE FOR BIOPOTENTIAL ELECTRODES (Attorney's Docket No. NEURO-12 PROV).

The above-identified patent application is hereby incorporated herein by reference.

### FIELD OF THE INVENTION

This invention relates to electrodes for monitoring electrical signals within the body and/or electrodes for electrically stimulating anatomical structures within the body, and more particularly to an integrated carrier for providing (i) support for an electrode array prior to use, (ii) templates for guiding proper placement of the electrode array onto the body of a patient, and (iii) instructions for use.

### BACKGROUND OF THE INVENTION

Biopotential electrodes are used extensively in the monitoring of electrical signals within the body. Biopotential electrodes are also used extensively to electrically stimulate anatomical structures.

In some circumstances, a plurality of biopotential electrodes are combined in a single electrode array, with one biopotential electrode being used to stimulate a first anatomical structure (e.g., a nerve) and a second biopotential electrode being used to monitor the resulting response by a second anatomical structure (e.g., a muscle). Since the stimulating electrode is applied to the first anatomical structure (e.g., over the peroneal nerve) and the monitoring electrode is applied to the second anatomical structure (e.g., over the extensor digitorum brevis muscle), the electrode array must frequently span substantial distances (e.g., 5 to 10 inches) and may need to be positioned around intervening anatomical structures (e.g., the ankle).

Electrode arrays of the sort described above are generally constructed on a single substrate formed from a flexible material such as MYLAR®, with electrical traces being formed on the flexible MYLAR® substrate. The stimulating and monitoring electrodes are typically located on separate sections of the substrate, with the several sections of the substrate being connected together by a thin strip of the substrate material. Since there is typically a single electrical connector for the entire electrode array, the aforementioned electrical traces must generally extend along the thin connecting strip of the electrode array.

Due to the flexible nature of the electrode array in general, and particularly to the highly flexible nature of the thin connecting strip of the electrode array, it is generally important to keep the electrode array mechanically stabilized during shipment and storage so as to prevent kinking of the thin connecting strip (and its associated electrical traces) and to prevent the different sections of the electrode array from adhering to one another. This is typically accomplished by mounting the electrode array on a carrier sheet after manufacture and prior to shipping.

In addition to the foregoing, a given electrode array is generally designed to be used for a specific application. For example, nerve conduction studies for the peroneal nerve are generally conducted by placing the stimulating electrode over the peroneal nerve and the monitoring electrode over the extensor digitorum brevis muscle, with the thin connecting strip connecting the two electrode sections. Thus, it is important that the healthcare provider properly position the electrode array on the patient in order to achieve accurate results. This is commonly done by first locating one or more physical landmarks on the body of the patient, then marking their position, and finally placing the stimulating and monitoring electrodes over the appropriate anatomy using the markings as a guide.

Thus, it is generally desirable for the electrode array manufacturer to provide guides to facilitate proper positioning of the electrodes on the patient. These guides are typically provided in the form of marking templates for placement against the patient's anatomy.

In addition, it is also generally desirable for the electrode array manufacturer to provide instructions to facilitate proper positioning of the electrodes on the patient. The instructions are typically provided in the form of an instruction sheet.

In summary, current electrode arrays generally require (i) a carrier sheet to provide support during shipping and storage, (ii) templates for guiding proper electrode placement during use, and (iii) instructions for use.

The manufacturers of electrode arrays currently provide a separate means to accomplish each of the functions identified above. More particularly, electrode arrays are generally provided with: (i) a carrier sheet pre-punched with several holes to receive the various sections of the electrode array, and tabs to hold the different sections in place, (ii) marking templates to guide proper placement of the electrode array during use, and (iii) an instruction card for providing instructions for use.

The provision of several separate means adds to the overall cost of the electrode array product and reduces profit margins in an increasingly cost-conscious healthcare environment.

It is, therefore, a principal object of the present invention to provide an integrated carrier which simultaneously provides (i) mechanical support for the electrode array during shipment and storage, (ii) marking templates to guide proper placement of the electrode array during use, and (iii) instructions for use.

### SUMMARY OF THE INVENTION

These and other objects of the present invention are addressed by the provision and use of an integrated carrier for a biopotential electrode array, the integrated carrier comprising:

a substantially planar, sheet-like body which is formed out of a material which is sufficiently stiff as to provide mechanical support to the electrode array during shipment and storage;

at least one securing unit disposed on the body for securing the biopotential electrode array to the body; and

at least one template disposed in the body for guiding proper placement of the electrode array against the patient.

In another form of the invention, there is provided an integrated carrier for a biopotential electrode array, the integrated carrier comprising:

a substantially planar, sheet-like body which is formed out of a material which is sufficiently stiff as to provide mechanical support to the electrode array during shipment and storage;

at least one securing unit disposed on the body for securing the biopotential electrode array to the body;

at least one template disposed in the body for guiding proper placement of the electrode array against the patient; and

instructions for use, wherein the instructions are printed on the body.

### BRIEF DESCRIPTION OF THE DRAWINGS

These and other objects and features of the present invention will be more fully understood from the following detailed description of the preferred embodiments of the invention, which is intended to be read in conjunction with the accompanying drawings, wherein like numbers refer to like parts and further wherein:

FIG. 1 is a schematic view showing the outer side of an electrode array;

FIG. 2 is a schematic view showing the inner side of the electrode array shown in FIG. 1;

FIG. 3 is a schematic view showing a first embodiment of the integrated carrier of the present invention;

FIG. 4 is a schematic view showing the electrode array of FIGS. 1 and 2 mounted to the integrated carrier of FIG. 3;

FIG. 5 is a schematic view showing a second embodiment of the integrated carrier of the present invention; and

FIG. 6 is a view like that of FIG. 5, except showing a portion of the integrated carrier being removed.

### DETAILED DESCRIPTION OF THE PREFERRED EMBODIMENTS

### The Electrode Array

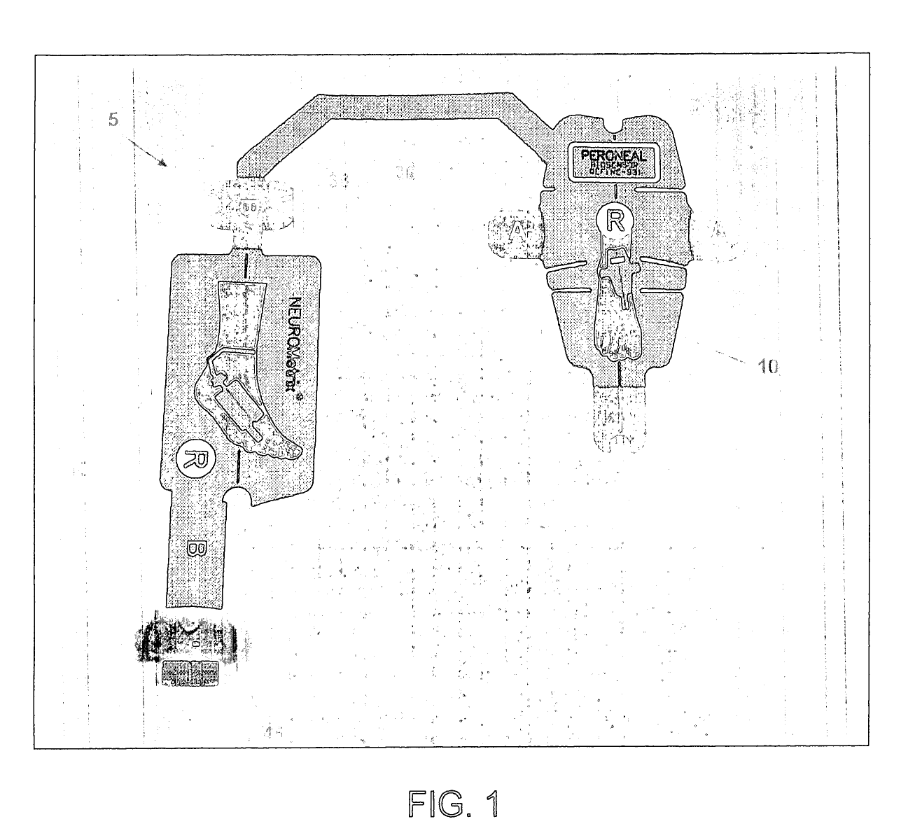

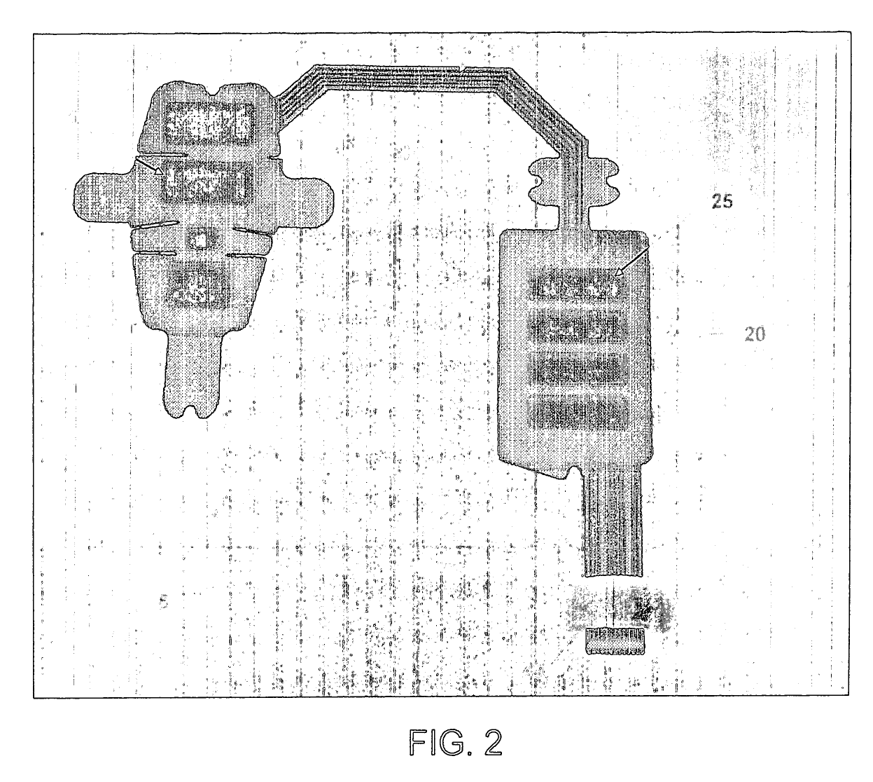

Looking first at FIGS. 1 and 2, there is shown an electrode array 5 which may be used in conjunction with the present invention. Electrode array 5 generally comprises a first section 10 comprising at least one stimulating electrode 15, a second section 20 comprising at least one monitoring electrode 25, and a thin connecting strip 30 for connecting first section 10 with second section 20.

Electrode array 5 is preferably formed from a single substrate 35 which is itself formed from a flexible material, e.g., MYLAR®. Electrical traces 40 extend through substrate 35 (including thin connection strip 30) and connect the at least one stimulating electrode 15 and the at least one monitoring electrode 25 with a connector 45. Connector 45 permits electrode array 5 to be connected to nerve conduction testing apparatus (not shown) of the sort well known in the art, i.e., apparatus for applying an appropriate electrical current to the at least one stimulating electrode 15 so as to stimulate a first anatomical structure (not shown), and apparatus for monitoring the resulting electrical response detected from a second anatomical structure (not shown) using the at least one monitoring electrode 25.

As is well known in the art, the at least one stimulating electrode 15, the at least one monitoring electrode 25, and the electrical traces 40 may be formed by silk screening, chemical plating or other conventional means for adding these elements to the flexible substrate 35.

As is also well known in the art, a conductive gel (not shown) is preferably positioned on the “skin side” of the electrodes so as to facilitate electrical contact between the electrodes and the skin of the patient.

And as is also well known in the art, an adhesive (not shown) applied to the inner side of the electrode array attaches the electrodes to the skin of the patient.

### The Integrated Carrier

The present invention comprises an integrated carrier for providing support, templates and instructions for electrode array 5.

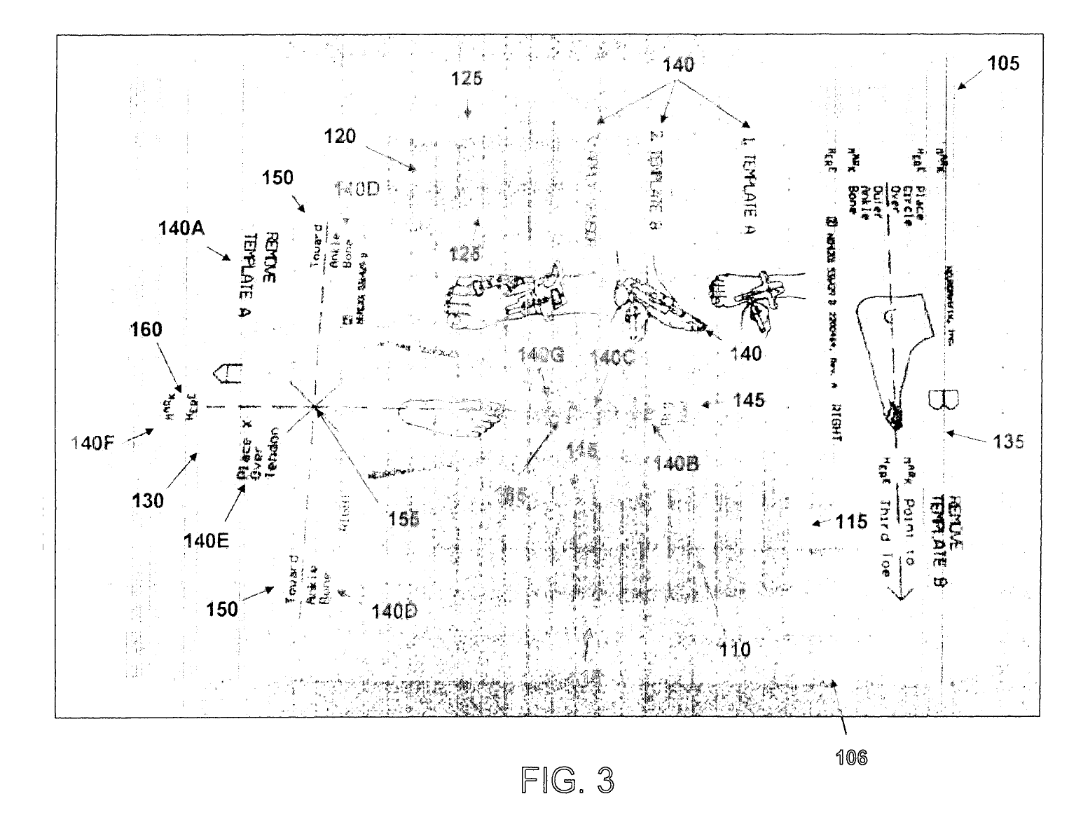

Looking now at FIG. 3, the present invention comprises an integrated carrier 105 for, among other things, receiving electrode array 5. Integrated carrier 105 comprises a substantially planar, sheet-like body 106 which is formed out of a material which is sufficiently stiff as to provide mechanical support to the electrode array during shipment and storage. Sheet-like body 106 is preferably transparent, although it may also be translucent or opaque or a combination of the foregoing. By way of example, in many situations it may be desirable for the sheet-like body 106 to yield templates (see below) which are see-through. In this case, it may be desirable to form at least part of the sheet-like body 106 out of a transparent or translucent material. The material(s) used to form sheet-like body 106 may also be selected based upon aesthetic considerations. By way of further example but not limitation, integrated carrier 105 may be constructed from plastic (e.g., MYLAR®) or paper or other suitable material. The thickness of integrated carrier 105 may depend on the particular material used and the overall stiffness desired. By way of example but not limitation, where integrated carrier 105 is formed out of MYLAR®, the carrier may have a thickness of between 0.005 inches and 0.015 inches.

1. Support. Still referring now to FIG. 3, integrated carrier 105 comprises an opening 110 for receiving first section 10 of electrode array 5. A plurality of tabs 115 are provided for holding first section 10 of electrode array 5 to integrated carrier 105, as will hereinafter be discussed. Integrated carrier 105 also comprises a second opening 120 for receiving second section 20 of electrode array 5. A plurality of tabs 125 are provided for holding second section 20 of electrode array 5 to integrated carrier 105, as will hereinafter be discussed.

If desired, additional tabs (not shown) may be provided on the integrated carrier 105 to hold thin connecting strip 30 to the integrated carrier.

If desired, integrated carrier 105 may omit opening 110 and/or second opening 120, so that electrode array 5 lies against the face of integrated carrier 105. The omission of opening 110 and/or second opening 120 may be more suitable in situations where a relatively thin electrode array 5 is to be mounted to integrated carrier 105.

2. Templates. Integrated carrier 105 also comprises a plurality of templates for guiding proper placement of electrode array 5 during use.

More particularly, in one preferred form of the invention, integrated carrier 105 comprises a template 130 for use in positioning first section 10 of electrode array 5 against the appropriate anatomy of the patient. This is done by first placing template 130 over a physical landmark on the body of the patient, then placing a mark on the anatomy of the patient, and then using that mark to position first section 10 of electrode array 5 against the anatomy of the patient, whereby to properly position stimulating electrode 15. To this end, template 130 is preferably die-cut into integrated carrier 105 so that the template may be selectively separated from the carrier when the template is to be used.

Similarly, in one preferred form of the invention, integrated carrier 105 comprises a template 135 for use in positioning second section 20 of electrode array 5 against the appropriate anatomy of the patient. This is done by first placing template 135 over a physical landmark on the body of the patient, then placing a mark on the anatomy of the patient, and then using that mark to position second section 20 of electrode array 5 against the anatomy of the patient, whereby to properly position monitoring electrode 25. To this end, template 135 is preferably die-cut into integrated carrier 105 so that the template may be selectively separated from the carrier when the template is to be used.

3. Instructions. Integrated carrier 105 also comprises instructions for use. More particularly, and still looking now at FIG. 3, integrated carrier 105 comprises instructions 140 (including graphics and text) to guide (i) removing templates 130, 135 from integrated carrier 105, (ii) placing templates 130, 135 on the body of the patient, (iii) appropriately marking the body of the patient to indicated where the several electrodes should be positioned, and (iv) applying the several electrodes to the body of the patient. By way of example but not limitation, instructions 140A instruct the user to remove template 130 from the integrated carrier; instructions 140B instruct the user to place toe 145 of template 130 over the foot of the patient so that arrow 140C points to the big toe of the patient; instructions 140D instruct the user to direct template wings 150 toward the ankle bones of the patient; and instructions 140E direct the user to place the “X” marking 155 over the tendon of the patient. Instructions 140F instruct the user to mark the patient at point 160; and instructions 140G instruct the user to mark the patient at point 165. Subsequently, the marking points 160 and 165 are used to position the electrodes on the body of the patient. Other instructions 140 are provided with respect to template 135, etc.

### Manufacture

The integrated carrier of the present invention is a novel construction that can be manufactured by employing, in a novel fashion, the simple and inexpensive manufacturing techniques commonly used in the industry, including those used to manufacture the electrode array itself.

In one preferred embodiment of the invention, instructions 140 are first printed on the carrier, preferably in a single printing pass. However, if desired, more than one printing pass may be used, e.g., if the same is required to print instructions of the required detail. In addition, different colors may be added for clarity or aesthetic reasons. It is important to note, however, that the cost of manufacturing increases with each printing pass.

Subsequently, the pre-printed carrier is die-cut, punched or laser cut, etc., preferably in one such manufacturing operation, so as to create simultaneously (i) holes 110, 120 and tabs 115, 125 which are used to hold electrode array 5 to the carrier, (ii) cut the perimeters of templates 130, 135, leaving small bridges so the templates remain intact during shipment and storage but may be easily torn-out by the healthcare provider during use, and (iii) trims the overall size.

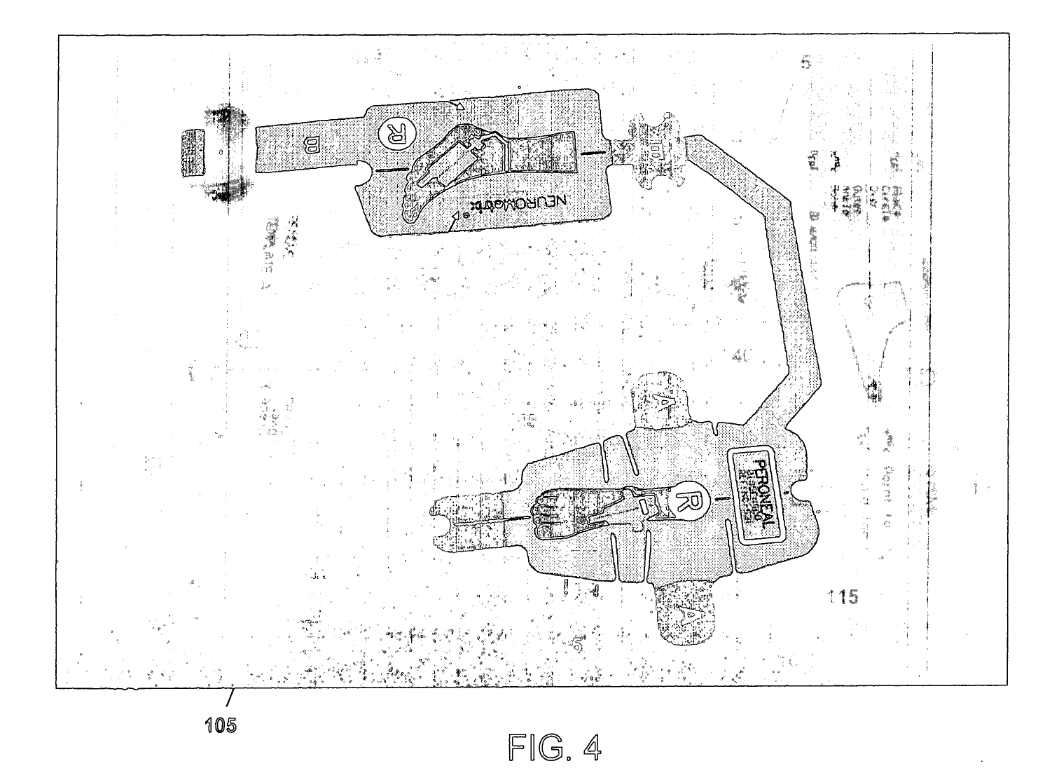

FIG. 4 shows an electrode array 5 mounted in integrated carrier 105. Note how tabs 115, 125 are slipped over the fronts of electrode sections 10 and 20 so as to releasably secure electrode array 5 to integrated carrier 105.

### Second Construction

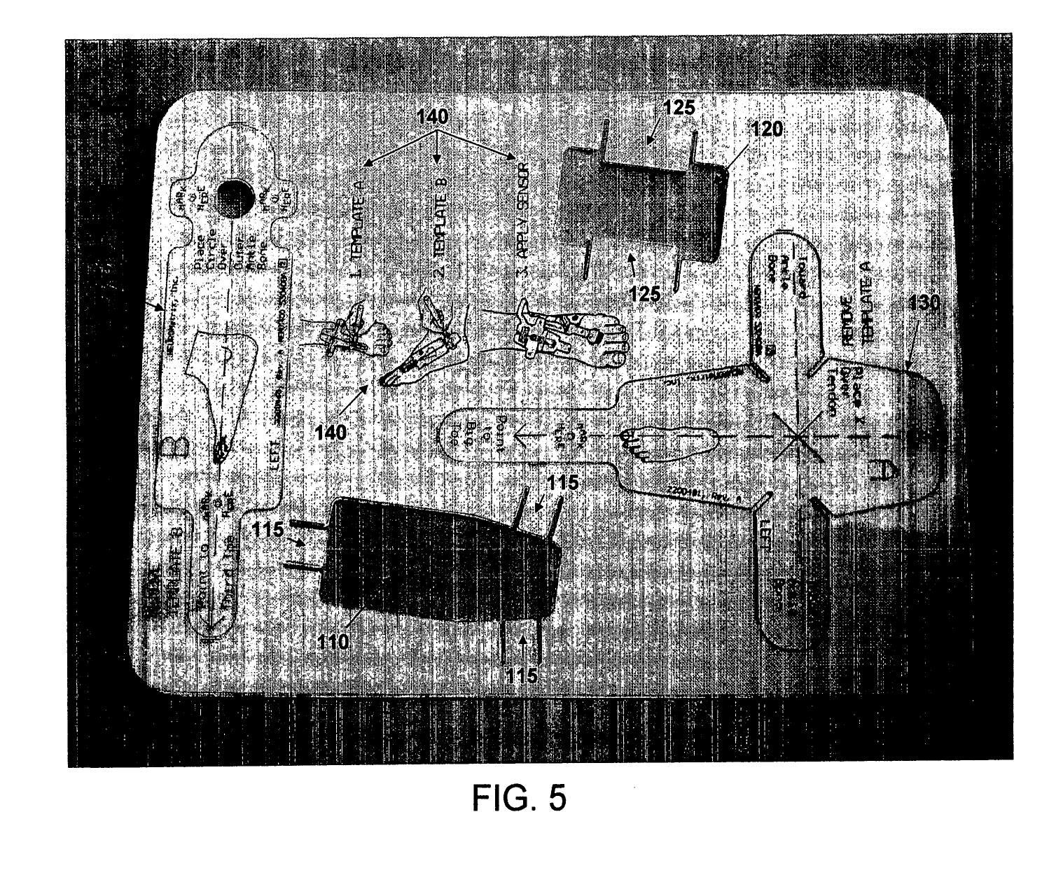

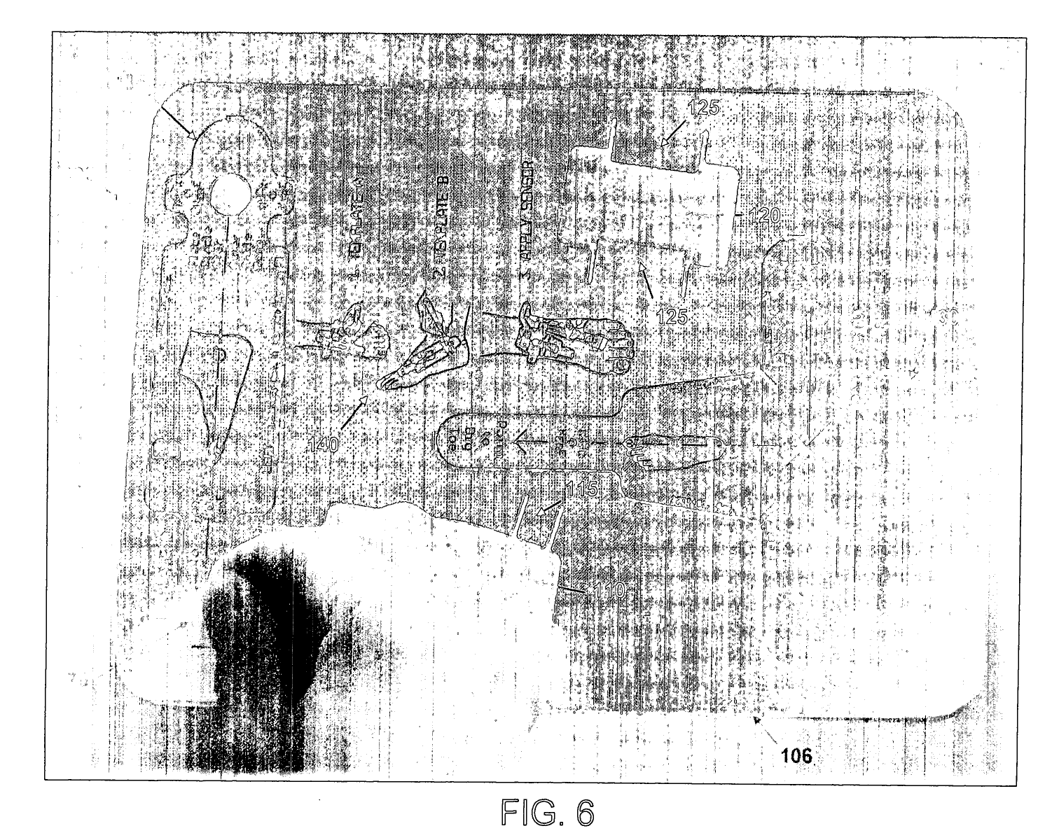

Looking next at FIGS. 5 and 6, in another embodiment of the present invention, an adhesive white background 170 is applied to the underside of integrated carrier 105. Adhesive white background 170 provides a contrast background for rendering instructions 140 more visible to the user when sheet-like body 106 is formed out of a transparent or translucent material. Adhesive white background 170 may be effected in various ways which will be known to those skilled in the art in view of the present disclosure, e.g., adhesive white background 170 may be formed using a release liner. By way of example but not limitation, adhesive white background 170 may be similar to a “crack-and-peel” label.

In the case where an adhesive white background is used, it may be desirable to form openings 110, 120 and tabs 115, 125 with a kiss-cut operation, rather than a standard die-cut operation, so that the adhesive white background is left intact after cutting of the foreground carrier.

### Third Construction

In another embodiment (not shown) of the present invention, electrode array 5 is held in place on integrated carrier 105 with low-tack surface adhesive instead of with openings 110, 120 and tabs 115, 125. The printing and die-cut (or kiss-cut) operations are similar to the previous embodiments.

### Modifications

While the foregoing invention has been described with reference to its preferred embodiments, various alterations and modifications will occur to those skilled in the art in view of the present disclosure. All such alterations and modifications are intended to fall within the scope of the invention.

What is claimed is:

1. An integrated carrier for a biopotential electrode array, the integrated carrier comprising:
a substantially planar, sheet-like body which is formed out of a material which is sufficiently stiff as to provide mechanical support to the electrode array during shipment and storage;
at least one securing unit disposed on the body for securing the biopotential electrode array to the body; and
at least one template disposed in the body for guiding proper placement of the electrode array against the patient.

2. An integrated carrier according to claim 1 wherein the body comprises plastic.

3. An integrated carrier according to claim 2 wherein the body comprises MYLAR®.

4. An integrated carrier according to claim 1 wherein the body comprises paper.

5. An integrated carrier according to claim 1 wherein the body is transparent.

6. An integrated carrier according to claim 1 wherein the body is translucent.

7. An integrated carrier according to claim 1 wherein the body is opaque.

8. An integrated carrier according to claim 1 wherein the body is colored.

9. An integrated carrier according to claim 1 wherein the securing unit comprises at least one tab.

10. An integrated carrier according to claim 9 wherein the at least one tab is cut out of the body.

11. An integrated carrier according to claim 10 wherein the at least one tab is cut out of the body using one method selected from the group consisting of punching, die-cutting and laser cutting.

12. An integrated carrier according to claim 1 wherein the securing unit comprises at least one hole.

13. An integrated carrier according to claim 1 wherein the securing unit comprises at least one hole, and at least one tab formed at the perimeter of the at least one hole.

14. An integrated carrier according to claim 1 wherein the securing unit comprises a light tack adhesive.

15. An integrated carrier according to claim 1 wherein the template is configured to be selectively separated from the carrier when the template is to be used.

16. An integrated carrier according to claim 10 wherein the at least one template is cut from the body using one method selected from the group consisting of punching, die-cutting and laser cutting.

17. An integrated carrier according to claim 1 wherein the carrier further comprises instructions for use, wherein the instructions are printed on the body.

18. An integrated carrier according to claim 1 further comprising a contrast sheet positioned against at least part of the body.

19. An integrated carrier for a biopotential electrode array, the integrated carrier comprising:
a substantially planar, sheet-like body which is formed out of a material which is sufficiently stiff as to provide mechanical support to the electrode array during shipment and storage;
at least one securing unit disposed on the body for securing the biopotential electrode array to the body;
at least one template disposed in the body for guiding proper placement of the electrode array against the patient; and
instructions for use, wherein the instructions are printed on the body.
